(19) United States
(12) Patent Application Publication
(10) Pub. No.: US 2021/0361498 A1
(43) Pub. Date: Nov. 25, 2021

(54) ABSORBENT ARTICLE WITH FOLDABLE INSERT

(71) Applicant: The Procter & Gamble Company, Cincinnati, OH (US)

(72) Inventors: Udo Friedel SCHOENBORN, Bad Soden (DE); Tonya Mae Sheldon WEST, Loveland, OH (US); Nicolette Johanna Birgitta Maria VANDERKLAAUW, Cincinnati, OH (US); Sara Lyn GIOVANNI, Cincinnati, OH (US)

(21) Appl. No.: 17/321,917

(22) Filed: May 17, 2021

Related U.S. Application Data
(60) Provisional application No. 63/028,021, filed on May 21, 2020.

(51) Int. Cl. A61F 13/84; A61F 13/15; A61F 13/536; A61F 13/56

(52) U.S. Cl. CPC A61F 13/84; A61F 13/15203; A61F 2013/8497; A61F 13/5616; A61F 13/536

(57) ABSTRACT

An absorbent insert includes a first lateral end and a second lateral end; and an adjustable attachment area. The adjustable attachment area includes an adjustment portion and an engageable portion, wherein the adjustment portion is capable of attaching a first segment of the absorbent insert to a second segment of the absorbent insert and wherein the engageable portion is capable of attaching the absorbent insert to an outer cover.

### FIELD OF THE INVENTION

The present invention relates generally to the field of wearable absorbent articles having absorbent inserts and reusable outer covers, and more particularly, systems for attaching the inserts to the outer covers.

### BACKGROUND OF THE INVENTION

It has long been known that absorbent articles (e.g., diapers, adult incontinence articles, feminine hygiene pads) offer the benefit of receiving and containing urine and/or other bodily exudates (e.g., feces, menses, mixture of feces and urine, mixture of menses and urine, etc.). It has been proposed to manufacture two-piece absorbent articles with a reusable outer cover and a detachable absorbent insert that may be reusable or disposable. In this way, the insert may be made with materials known to provide even more superior performance while minimizing the amount of waste as the whole article need not be disposed.

Unlike fully disposable absorbent articles, two-piece articles require the caregiver/user to properly place the insert into the outer cover. An insert may be too large for a given outer cover or to fit properly for the anatomy of the wearer. To address this issue, manufacturers may a create a number of different sized absorbent inserts; however, doing so requires additional production and material costs and may create complexity for the purchaser as he or she must determine the best option from multiple choices.

Therefore, there is a need for an adjustable absorbent insert that permits use for different sized wearers and/or different sized outer covers. There is also a need for making such absorbent inserts with minimum complexity and in a cost efficient manner.

### SUMMARY OF THE INVENTION

The invention comprises the features of the independent claims herein. An adjustable absorbent insert comprises a first lateral end and a second lateral end, and a length, L extending between the first and second lateral ends; and an adjustable attachment area. The adjustable attachment area comprises an outboard lateral edge disposed within 10 mm of the first lateral end and a length, AL1, that is between 20% and 70% of the length, L. The adjustable attachment also comprises an adjustment portion capable of attaching a first segment of the absorbent insert to a second segment of absorbent insert and an engageable portion. In an attached configuration, the engageable portion comprises a length, EL1, of at least 10 mm.

An absorbent article comprises an absorbent insert and an outer cover. The absorbent insert comprises a topsheet, a backsheet, and an absorbent core disposed between the topsheet and backsheet; a longitudinal axis and a lateral axis; and a first lateral end and a second lateral end. The absorbent article also comprises an adjustable attachment area having an adjustment portion and an engageable portion, wherein the adjustment portion is capable of attaching a first segment of the absorbent insert to a second segment of the absorbent insert and wherein the engageable portion is capable of attaching the absorbent insert to the outer cover.

An absorbent article comprises an absorbent insert and an outer cover. The absorbent insert comprises a topsheet, a backsheet, and an absorbent core disposed between the topsheet and backsheet; a longitudinal axis and a lateral axis; a first lateral end and a second lateral end, and a length, L extending between the first and second lateral ends. The absorbent insert also comprises a first adjustable attachment area having a length AL1, that is between 20% and 70% of L, wherein the first adjustable attachment area comprises an adjustment portion and an engageable portion. The adjustment portion is capable of attaching a first segment of the absorbent insert to a second segment of the absorbent insert and the engageable portion is capable of attaching the absorbent insert to the outer cover. The absorbent insert further comprises a second attachment area, wherein the first adjustable attachment area and the second attachment area are separated by a distance of at least 50 mm.

### BRIEF DESCRIPTION OF THE DRAWINGS

FIG. 1 is a perspective view of a wearable absorbent article as it might appear being worn by a wearer about the lower torso;

FIG. 2 is a plan view of an outer cover opened and laid flat, inner surface facing the viewer;

FIG. 3 is a plan view of an absorbent insert opened and laid flat, outer (garment-facing) surface facing the viewer;

FIG. 4 is a perspective view of a disposable absorbent insert shown apart from an outer cover, as it might appear in a free-standing, relaxed state;

FIG. 5 is a plan view of a disposable absorbent insert shown stretched out and laid flat, wearer-facing surfaces facing the viewer;

FIG. 6 is a cross sectional view of an example of an absorbent insert such as shown in FIG. 5, taken at line 6-6 in FIG. 5;

FIGS. 7A-7B are schematic lateral cross sectional views of examples of absorbent inserts having a dual leg gasketing system, taken through a lateral axis of the insert example;

FIG. 8A is a plan view of an absorbent insert opened and laid flat, garment-facing surface facing the viewer;

FIG. 8B is a plan view of the absorbent insert of FIG. 8A in an attached configuration, garment-facing surface facing the viewer;

FIG. 9A is a plan view of an absorbent insert opened and laid flat, garment-facing surface facing the viewer;

FIG. 9B is a plan view of the absorbent insert of FIG. 9A in an attached configuration, garment-facing surface facing the viewer;

FIG. 10A is a plan view of an absorbent insert opened and laid flat, garment-facing surface facing the viewer; and

FIG. 10B is a plan view of the absorbent insert of FIG. 10A in an attached configuration, garment-facing surface facing the viewer.

### DETAILED DESCRIPTION OF THE INVENTION

“Absorbent article” means a device that absorbs and contains body exudates and, more specifically, devices that are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, and the like.

“Absorbent insert” and “insert” mean a component of a wearable absorbent article that is adapted to contain and/or absorb urine, feces, menses or any combination thereof, and is adapted to be installable and removable as a modular unit, from an outer cover.

“Disposed” refers to an element being located in a particular place or position. A feature that is disposed on a surface or side of a component may be integral with said component or may be joined to said component.

“Disposable”, when referring to an absorbent insert, means that the absorbent insert is not adapted or intended to be effectively sanitarily laundered in an ordinary household laundering process and ordinary household equipment, and thereby is ordinarily unsuitable for sanitary and effective reuse so as to provide as-new intended functions and performance, following soiling by exudates and removal from an outer cover. By way of nonlimiting examples, effective laundering may be frustrated or prevented, causing the insert to be disposable, by inclusion of materials and/or construction: that do not retain their substantial as-new physical shape or structure through ordinary household laundering and drying so as to be effective as-new in reuse; that absorb aqueous liquids and cannot be sufficiently dried/dehydrated in ordinary household drying equipment and ordinary drying cycles so as to be effective as-new in reuse; that dissolve or substantially degrade in ordinary household laundering or drying, causing the insert to be substantially damaged or rendered useless; and/or that cannot be effectively cleaned of exudate material through ordinary laundering, so as to be sanitary and otherwise acceptable for re-use.

“Inboard,” with respect to a first feature of an article and its position relative a second feature or location on the article, means that the first feature lies closer to a respective axis of the article than the second feature or location, along a horizontal x-y plane approximately occupied by the article when laid out flat, extended to the full longitudinal and lateral dimensions of its component web materials against any contraction induced by any included pre-strained elastomeric material, on a horizontal surface. Laterally inboard means the first feature is closer to the longitudinal axis, and longitudinally inboard means the first feature is closer to the lateral axis. Conversely, “outboard,” with respect to a first feature of an article and its position relative a second feature or location on the article, means that the first feature lies farther from the respective axis of the article than the second feature or location.

“Joined” or “attached” means configurations whereby an element is directly secured to another element by affixing the element directly to the other element, and configurations whereby an element is indirectly secured to another element by affixing the element to intermediate member(s) that in turn are affixed to the other element.

“Longitudinal” means a direction lengthwise in a component such that the longitudinal direction runs parallel to the maximum linear dimension in the x-y plane of the component. In an absorbent article as described herein, the longitudinal direction runs substantially perpendicular from a waist end edge to an opposing waist end edge when the absorbent article is in a flat out, uncontracted state, or from a waist end edge to the bottom of the crotch in a bifolded article.

“Lateral” refers to a direction generally perpendicular to the longitudinal direction. In the absorbent article described herein, the lateral direction runs substantially parallel from a side edge to an opposing side edge.

“Outer cover” means a component of a wearable absorbent article that is adapted to be worn about the lower torso of a wearer, and is adapted to support or in normal use is capable of supporting and holding an absorbent insert. The term encompasses a wrapping structure (such as included in a conventional diaper) and a pant structure (such as included in underwear for the lower torso, of any design).

“Reusable”, when referring to a component means that the component is adapted to be used for its intended purpose after initial use without substantial destruction of any portions of the component necessary for as-new functionality. For example, a reusable outer cover means an outer cover that is adapted to permit removal of at least a first insert, and replacement thereof with at least a second insert, without substantial destruction of any portions of the outer cover that are necessary to provide the substantial as-new functionality of the outer cover, and without the necessity of any repair or reconstruction following such insert replacement.

### Overview

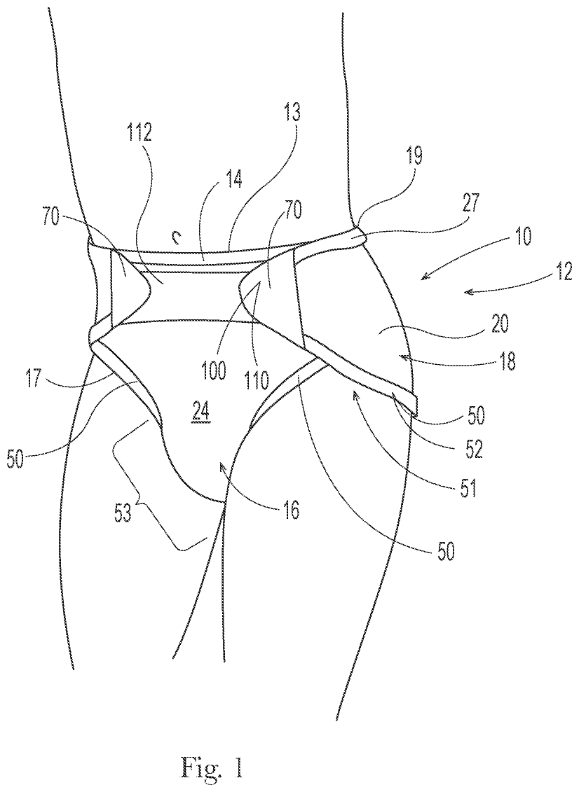

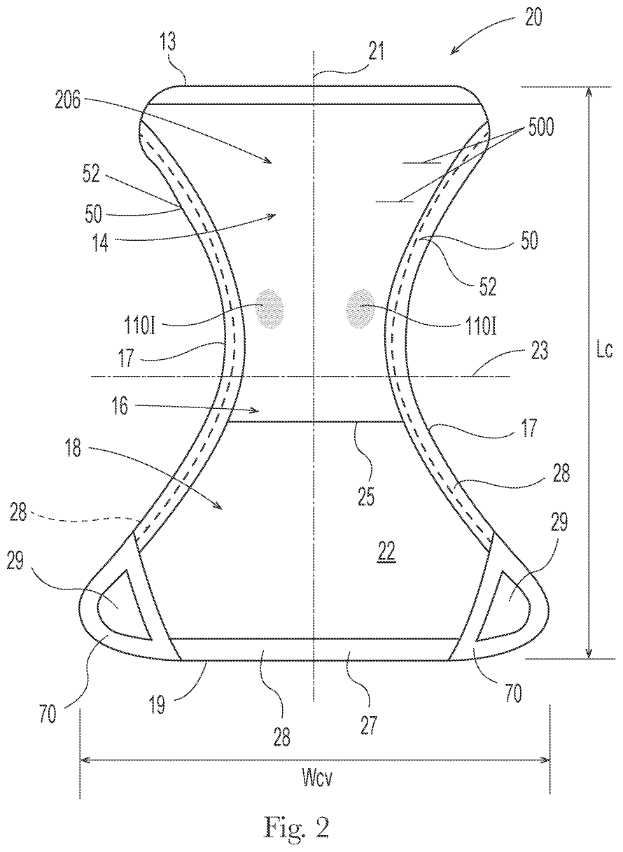

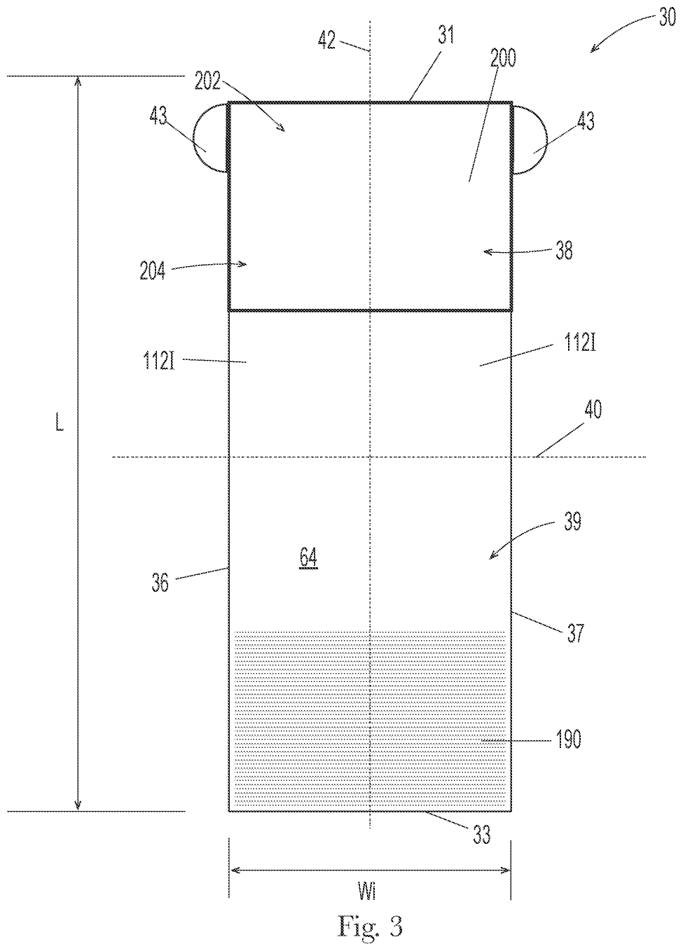

FIG. 1 is a perspective view an exemplary, nonlimiting embodiment of an absorbent article 10. The article is shown in the form of a two-piece absorbent article 12, having an outer cover 20 and an absorbent insert 30 shown in FIGS. 2-3. It is to be understood that during manufacturing, the article may comprise several discrete pieces that are joined together. However, by two-piece absorbent article, it is meant that the article in its final form has two components that the user assembles together for wear. While shown as a two-piece absorbent article, it is also contemplated that the article may comprise more than two pieces, such as attachable ears or belts. Returning to FIG. 1, it can be seen that the absorbent article 10 may have a front region 14, a rear region 18 and a crotch region 16 disposed between the front and rear regions. The article may be placed on a wearer by wrapping the outer cover 20 between the wearer's legs and under the buttocks such that the crotch region 16 is between the wearer's legs. When the insert 30 has been installed onto the outer cover 20, the insert 30 will then be disposed next to the wearer. Nonlimiting examples of two-piece articles are disclosed in U.S. Pat. Nos. 8,998,870, 9,089,456, 8,435,223, 9,011,402, 8,808,263, 8,759,605 and 8,932,273 and 9,078,789. In various embodiments, the outer cover is reusable and the absorbent insert is disposable.

As shown in FIG. 3, the absorbent insert includes one or more adjustable attachment areas 200 that comprises an adjustment portion 202 and an engageable portion 204. The adjustment portion 202 is capable of attaching the absorbent insert to itself while the engageable portion is capable of attaching the absorbent insert to the outer cover. These and other features will be further described below.

Outer Cover FIG. 2 depicts an exemplary outer cover 20 as it may appear opened and laid flat. In FIG. 2, the wearer-facing surface of the outer cover 20 faces the viewer. The outer cover comprises a front lateral edge 13, a rear lateral edge 19, and two longitudinal edges 17. The outer cover comprises a length, Lc, from the outboard-most portion of the front lateral edge to the outboard-most portion of the rear lateral edge. The outer cover may comprise a lateral axis 23, dividing evenly its length, and a longitudinal axis 21. The longitudinal edges 17 may be parallel to the longitudinal axis 21. However, for better fit, longitudinal edges 17 may be curved or angled to produce, for example, an “hourglass” shape article when viewed in a plan view as shown in FIG. 2, for example. In nonlimiting examples, the outer cover may be asymmetric, for instance having a width its rear region that is greater than the width in its front region. Thus, the width of the outer cover, Wcv, as measured between the longitudinal edges along a line parallel to the lateral axis, may vary along the longitudinal length of the outer cover. Nonlimiting examples of outer covers are disclosed in U.S. Pat. Nos. 9,387,138 and 8,435,223.

If it is desired that outer cover 20 be reusable, for outer cover 20 to remain substantially sanitary and useful (without requiring laundering or disposal) after removal and replacement of an insert, it may be desired that all parts of outer cover 20 remain substantially unsoiled after an exudation of waste (especially fecal matter) by the wearer. Thus, it may be desired that when insert 30 is installed within an outer cover 20, there is no non-removable portion or component of outer cover 20 that lies over or covers a substantial portion of wearer-facing surfaces of insert 30. Expressed differently, no non-removable portion or component of outer cover 20 is situated between a substantial portion of insert 30 and the wearer when the wearable absorbent article is worn, at least in the areas proximate to wearer body features that discharge exudates. Thus, it may be desired that outer cover 20 include no non-removable cover sheet or the like that covers or contains substantial portions of wearer-facing surfaces of insert 30 within outer cover 20, nor any overlying structures such as pockets, straps or flaps that substantially wrap or cover the insert proximate to exudate discharge points, or lie substantially between insert 30 and the wearer's anus and/or genitals, when the wearable absorbent article is worn. If outer cover 20 lacks such overlying structures, this may increase the likelihood that the wearer's exudates will contact only insert 30, and not portions of outer cover 20.

Outer cover 20 and/or layers or portions thereof may be made of any durable or semi-durable knitted, woven or nonwoven textile or textile-like material that is appropriately compatible with skin of the intended wearer(s). Suitable examples are described in U.S. application Ser. Nos. 12/687,493; 12/687,412; 12/687,528; and Ser. No. 12/687,425 (all by Roe et al.).

Non-limiting examples of fibers, nonwovens and laminates of nonwovens and films that might be considered for use as semi-durable outer cover materials may be found in U.S. Pat. Nos. 7,223,818; 7,211,531; 7,060,149; 6,964,720; 6,905,987; 6,890,872; 6,884,494; 6,878,647; and 5,518,801; and U.S. Published Applications Nos. 2008/0319407; 2008/0045917; 2007/0293111; 2007/0287983; 2007/0287348; 2007/0249254; 2007/0203301; and 2005/0164587.

FIG. 1 depicts an exemplary outer cover, with its garment-facing surface 24 facing the viewer. In nonlimiting examples, the garment-facing surface 24 of the outer cover may be formed by a first layer of a durable or semi-durable material. The material selected may include fibers having hydrophobic properties, providing enhanced liquid containment attributes to such first layer. In another example, however, it may be desirable in some circumstances for the selected material to include hydrophilic fibers, or fibers treated to be hydrophilic, so as will cause the material to more readily absorb and/or transmit liquid therethrough. This may serve to provide supplemental absorbency within the outer cover for the event in which liquid exudates escape the insert, or to provide one way of communicating to the user that liquid exudates have escaped the insert. Additionally, in some circumstances, it may be desirable that the material selected have soft tactile properties so as to have a pleasant feel that the user and/or wearer find attractive. The material also may be selected so as to have a desired appearance, including but not limited to coloration, sheen, and/or texture.

Outer cover 20 may be formed of a single layer of a durable or semi-durable material, or may have two or more layers in the front region 14 and/or rear region 18. Accordingly, referring to FIG. 2, in nonlimiting examples, a wearer-facing surface 22 of the outer cover may be formed by a second layer of a durable or semi-durable material. The material selected may include fibers having hydrophobic properties, providing enhanced liquid containment attributes to the second layer. In another example, however, it may be desirable in some circumstances for the selected material to include hydrophilic fibers, or fibers treated to be hydrophilic. This may be desired in some circumstances to cause the material forming inner surfaces 22 to more readily absorb liquid, or transmit liquid therethrough. This may serve to provide supplemental absorbency within the outer cover for an event in which liquid exudates escape the insert, reducing the likelihood that the outer cover will leak. Alternatively, it may provide one way of communicating to the user that liquid exudates have escaped the insert, by causing wetness to be transmitted through to the outer cover outer layer such that wetness is visible on outer surfaces. Alternatively, it may serve to provide a layer that tends to draw moisture away from the skin, for a drier, more comfortable feel.

Additionally, in some circumstances, it may be desirable that the material(s) selected for inner surfaces 22 have soft tactile properties so as to have a pleasant feel against the skin, particularly in areas where no portion of an insert is expected to be present between the outer cover and the wearer's skin. Further, it may be desirable that at least a portion of the inner surface comprise a material (e.g., comprising loops) that is engageable by hook fastening components. Additionally, or alternatively, a second layer of material may be formed of a textile material having enhanced elasticity, such as by inclusion of fibers of an elastomeric material (such as spandex). In another example, an intermediate film layer may be included, laminated or not laminated with another layer.

In addition to forming differing layers of differing materials, it may be desirable to form a single layer of differing materials, for example, differing materials in the respective front, crotch and/or rear regions of the outer cover. Such differing materials may be joined at a seam 25, which may be formed on wearer-facing surface and/or the garment-facing surface. For example, the material predominately forming the inner surface of rear region 18 may be selected primarily for its elasticity features, which may better serve to provide snug fit about wearer body contours and accommodate wearer movement (i.e., about the buttocks and hips). By comparison, the material predominately forming the inner surface of front region 14 and/or crotch region 16 might be selected primarily for its hydrophobicity or hydrophilicity, which may better serve to contain liquid exudates.

Layers or other elements of the outer cover may be joined to each other via any suitable mechanism, including, for example, adhesives, mechanical bonding, ultrasonic bonding, sewing, stitching, serging, edging, and the like.

Waist Features, Leg Gasketing Systems, Ears

Referring again to FIGS. 1-2, the article and outer cover may comprise one or more waist features 27. The waist feature may be elastic and thereby provide better fit about the waist of the wearer. Elasticized waist features include waistbands, waist cuffs having pockets formed from a portion of the waist feature that is unattached from the remainder of the outer cover, and waist panels and/or belts designed to fit securely about the abdomen of the wearer. Nonlimiting examples of elasticized waist features are disclosed in U.S. patent application Ser. Nos. 14/533,472; 15/074,675 and 62/855,001. Elasticized waist features may comprise one or more nonwoven or textile layers, which may be layers of the outer cover or discrete portions, and one or more elastic elements 28. In nonlimiting examples, the elasticized waist feature comprises elastic strands joined to the nonwoven and/or textile layer(s). In further nonlimiting examples, the elasticized waist feature comprises a laminate of one or more textile and/or nonwoven layers and one or more films. The elasticized waist element may comprise one or more rugosities if the elastic material is strained prior to lamination. In other nonlimiting examples, the layers of the elastic laminate may be joined at zero applied strain and subsequently activated.

In alternative embodiments, the waist feature may be inelastic. In such configurations, the waist feature may provide additional anchoring about the waist of the wearer.

Additionally, or alternatively, the article may comprise a leg gasketing system 50, portions of which may be formed by the outer cover and/or the absorbent insert. The outer cover may include one or more elastic elements 28, such as films or elastic strands, extending through all or a portion of the leg opening 51 to form a leg band portion 52. The elastic elements may be laminated with one or more nonwoven layers and/or one or more textile layers. As described with respect to the waist feature, the leg band portion may include rugosities as result of the lamination process. In other nonlimiting examples, the leg band portion may be a zero strain, activated laminate.

The waist features and/or leg band portions may be disposed along the edge of the outer cover, and in some circumstances, it may be desired to have elasticized waist and leg band portions situated along substantially the entire length of the leg and/or waist openings so as substantially or completely encircle the wearer's legs and/or waist while outer cover 20 is worn. The gathered material within rugosities can serve to accommodate stretching of waist feature and leg band portions. This arrangement not only may provide for better fit about the wearer's legs, but also may enable the outer cover 20, when formed of appropriately sized and shaped material, to form a pouch-like structure 53 in the crotch region (see FIG. 1) when worn, which may serve to provide space within the outer cover to accommodate the insert 30 and help hold it in place within outer cover 20, in a substantially laterally centered position within the crotch region. This may be deemed advantageous in examples in which an insert is attached within outer cover 20 by fastener components only located proximate to the respective ends of insert, and not at any longitudinally intermediate locations, as described further below. Alternatively, or additionally, the elastic strands or strips in waist features 27 and leg band portions 52 may be affixed within the outer cover only at or near their respective ends, e.g., within a pouch, tube or envelope structure formed of outer cover material—referred to herein as a “drawstring elastic”. This will allow the elastic material and associated outer cover material to stretch and move freely and independently of each other, which may promote fit and comfort. A snug fit about the wearer's legs provided by such elasticized leg band portions 52 may serve to enhance containment capability of the wearable absorbent article. The outer cover 20 may also include anchoring supplements, bands or systems thereof as described in more detail in U.S. Pat. No. 8,932,273.

The outer cover may include ears 70 in one or both of the front and back regions. As shown in FIGS. 1 and 2, the ears may include fastening components 110 such that the ears can be secured to the opposing region (e.g., rear ears may include fastening components that may engage with the front region). The outer cover may include receiving components 112 to operatively engage with fastening components 110. Nonlimiting examples of engageable fastening and receiving components include tape tabs, hook and loop fastening components, interlocking fasteners such as tabs & slots, buckles, buttons, snaps, and/or hermaphroditic fastening components. Some exemplary surface fastening systems are disclosed in U.S. Pat. Nos. 3,848,594; 4,662,875; 4,846,815; 4,894,060; 4,946,527; 5,151,092; and 5,221,274. An exemplary interlocking fastening system is disclosed in U.S. Pat. No. 6,432,098. A fastening component may be discrete from and joined to the outer cover or may be integral with the outer cover. The receiving component may be discrete from and joined to the outer cover or may be integral with the outer cover. The fastening component and/or the receiving component may further include a release tape or other material, including folded material, that protects the component from insult prior to use. In nonlimiting examples, a fastening component 110 is disposed on a wearer-facing surface 22 of the outer cover and a receiving component 112 is disposed on a garment-facing surface 24. In such nonlimiting examples, the outer cover may comprise two or more fastening systems having distinct fastening locations, which deconcentrates lateral tensile focuses and reduces the tendency of the front portion of the article to pivot around the single fastening location. Suitable dual fastening systems are disclosed for example in U.S. patent application Ser. Nos. 16/684,230 and 16/545,425.

The outer cover may also comprise one or more insert fastening components 110I capable of operatively engaging with an insert receiving component 1121 disposed on the absorbent insert, as shown in FIGS. 2-3. The insert fastening and receiving components may comprise any of the exemplary components disclosed.

The fastening and/or receiving components may be discrete from and joined to the outer cover or absorbent insert or may be integral with one or both. In nonlimiting examples, nonwoven material (such as nonwoven material forming portions of the outer cover surfaces or portions of the insert backsheet or topsheet) may be comprise integral loops material as illustrated in FIG. 3. Further to the above, it is to be understood fastening systems need not necessarily include respective components of a two-component fastening system. Rather, a fastening system may require only one component. By way of nonlimiting example, a fastener component on outer cover 20 may include a patch of adhesive; a structure having a region of relatively high coefficient of friction; a pocket 29; flap; strap; or other capturing, holding and/or retaining surface, device or structure. Thus, a receiving component is unnecessary. Referring to FIG. 2, in some nonlimiting examples, the outer cover 20 may include one or more pocket structures 29 situated on or along the inner surface 22. A pocket structure may be adapted to receive, fit and capture, a lateral edge and portion of the insert, for example, the forward edge and a portion of forward region 38.

The outer cover further comprises one or more receiving portions 206 that are operatively engageable with an engageable portion 204 of the absorbent insert as is discussed in more detail below.

### Absorbent Insert

Returning to FIG. 3, the absorbent insert 30 may be designed to contain and/or absorb body exudates, and may be made of pliable materials as will be described further below. The insert 30 includes a forward region 38 and a rearward region 39, a first lateral end 31 and a second lateral end 33, a first longitudinal edge 36, a second longitudinal edge 37, a lateral axis 40 and a longitudinal axis 42. The insert 30 comprises a length L from the outboard-most portion of front edge 31 to the outboard-most portion of the rear edge 33. The lateral axis equally divides the length L. In nonlimiting examples, the insert length L is less than the length of the outer cover Lc.

The longitudinal edges 36, 37 may be generally parallel to the longitudinal axis. Alternatively, the longitudinal edges 36, 37 may be curved, such as in an hour-glass configuration. Thus, the width of the insert, Wi, may vary. In nonlimiting examples, the width of the insert, Wi, is less than the width of the outer cover, Wcv, at one or more longitudinal positions. The width of the insert, Wi, may be less than the width of the outer cover, Wcv, throughout the length of the insert.

The insert also may include one or more grasp structures 43, which may be accessible from a longitudinal side and/or from a lateral side of the insert, for example. The grasp structures may be provided to enable the user to quickly and easily grasp the insert, handle the insert during application and/or properly place the insert. In certain embodiments, the grasp structure may have a different tactile feel than surrounding or adjacent areas to distinguish the area and ease the user's identification of the grasp structures.

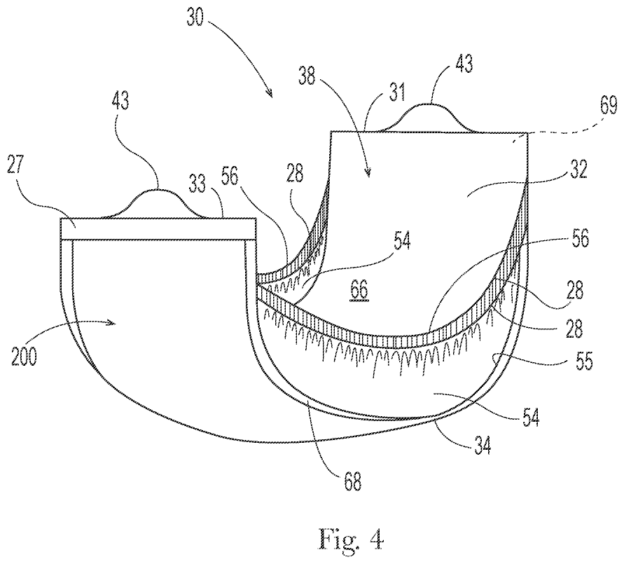

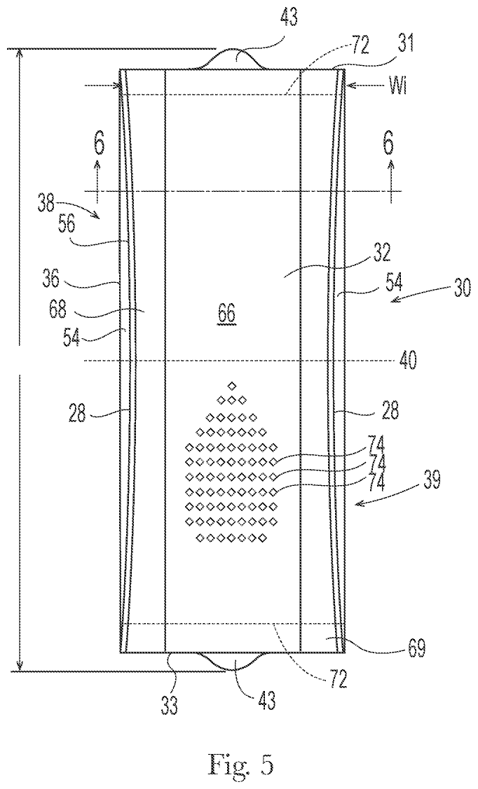

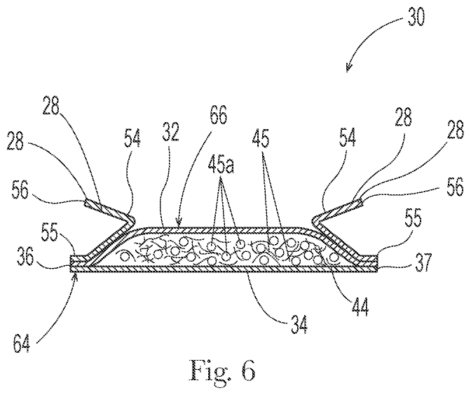

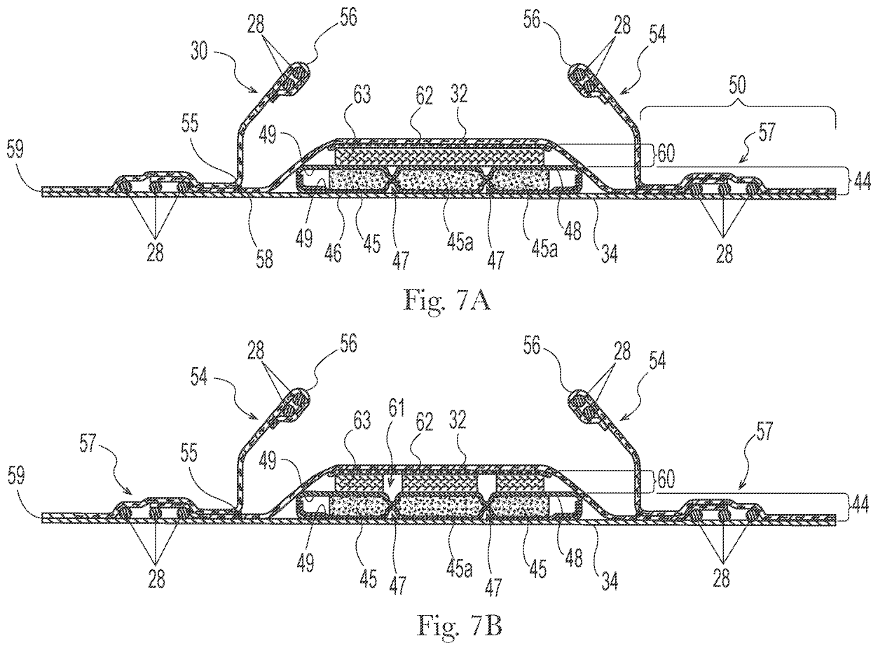

FIG. 4 depicts a disposable absorbent insert 30 in perspective view as it might appear in a free-standing, relaxed state, with both the body-facing surface 66 and garment-facing surface 64 shown. FIG. 5 depicts an example of an insert 30 shown stretched out and laid flat (against elastic-induced contraction to a position similar to that shown in FIG. 4), body-facing surface 66 facing the viewer. FIG. 6 depicts an exemplary cross-section of FIG. 5 according to certain nonlimiting examples. FIGS. 7A-7B depict lateral cross sectional views of examples of inserts having a dual leg gasketing system, taken through a lateral axis of the insert example.

As shown in FIGS. 4-5, the insert 30 may have a topsheet 32 and a backsheet 34. The topsheet and backsheet may be joined together along longitudinal seams 68 and along lateral seams 69. An absorbent core 44 may be disposed between the topsheet and the backsheet as shown for example in FIG. 6.

Returning to FIGS. 4-5, the insert 30 may further include longitudinal standing cuffs 54 affixed along the longitudinal sides 36, 37. Nonlimiting examples of absorbent inserts and details of their features are disclosed in U.S. Pat. Nos. 8,546,641 and 9,011,402.

It may be desirable to include a stiffening component 72 proximate to one or both ends of the insert 30, but especially an end adapted with a singularized and relatively localized fastening location providing a force-decoupled arrangement, as is discussed below. Referring to FIG. 5, end support stiffeners 72 may be included. Such end support stiffeners may serve to aid the user in engaging the insert with the outer cover and to help insert 30 maintain its intended shape and configuration while being worn beneath an outer cover (i.e., help maintain the intended position and gasketing function of the standing cuffs 54 which are discussed below). End support stiffeners 72 also may help control the corners of the insert regardless of the size, type or location of fastener components included on the insert. End support stiffeners 72 may provide resistance to bending in any direction or plane. Further, because the ends of insert 30 may otherwise be folded over or bunched while being stored and/or carried by the user before installation, end support stiffeners may enhance user convenience, by causing the ends of insert 30 to maintain or seek a shape/configuration that requires less manipulation by the user to install it in an outer cover.

Top Sheet

The topsheet 32 is generally a portion of the absorbent article 10 that may be positioned at least in partial contact or close proximity to a wearer. Suitable topsheets 32 are generally supple, soft feeling, and non-irritating to a wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured, may have any suitable three-dimensional features, and/or may have a plurality of embossments (e.g., a bond pattern). The topsheet 32 may comprise one or more apertures 74. The topsheet may be apertured by overbonding a material and then rupturing the overbonds through ring rolling, such as disclosed in U.S. Pat. No. 5,628,097, to Benson et al., issued on May 13, 1997 and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

Topsheet 32, backsheet 34 or any portion of the top sheet or backsheet may be embossed and/or matte finished to provide a more cloth-like appearance.

Backsheet

Backsheet 34 is generally the outer liner portion of insert 30 forming the garment-facing surface 64 thereof, and prevents the exudates absorbed and contained within insert 30 from wicking through and soiling the outer cover.

The backsheet 34 may comprise one or more nonwovens, elastomeric films, foams, strands, or combinations of these or other suitable materials with nonwovens or synthetic films. In nonlimiting examples, the backsheet is a laminate of an elastomeric material, such as a film and a nonwoven.

In various embodiments, the backsheet 34 is substantially water-impermeable. The backsheet may, for example, be or comprise a thin plastic film, such as a thermoplastic film having a thickness of about 0.012 mm to about 0.051 mm. Other suitable backsheet 34 materials may include breathable materials that permit vapors to escape from the absorbent article 10 while still preventing exudates from passing through the backsheet 34.

In certain embodiments, the backsheet 34 may have a water vapor transmission rate (WVTR) of greater than about 2000 g/24 h/m², greater than about 3000 g/24 h/m², greater than about 5000 g/24 h/m², greater than about 6000 g/24 h/m², greater than about 7000 g/24 h/m², greater than about 8000 g/24 h/m², greater than about 9000 g/24 h/m², greater than about 10000 g/24 h/m², greater than about 11000 g/24 h/m², greater than about 12000 g/24 h/m², greater than about 15000 g/24 h/m², measured according to WSP 70.5 (08) at 37.8° C. and 60% Relative Humidity. A higher WVTR may be desired in this particular application, since the insert backsheet 34 will not form the outer surface of the wearable article, as a conventional disposable diaper backsheet would, but rather, will be covered by the one or more layers of the outer cover material(s)—which themselves may act in some circumstances to reduce WVTR of the composite structure.

Other suitable materials and/or manufacturing techniques may be used to provide a suitable backsheet 34 including, but not limited to, surface treatments, particular film selections and processing, particular filament selections and processing.

Backsheet 34 may be joined to topsheet 32, absorbent core 44 or any other element of insert 30 by any suitable attachment mechanism known in the art.

It will be appreciated that the outer cover described above can be constructed of materials and construction so as to bear and sustain a majority of the structural loading generally imposed upon a disposable diaper, by stretching and accommodation of the wearer's anatomical features and body movements, and by absorption, swelling and added weight resulting from the wearer's exudations of waste. Thus, lesser requirements for structural strength of an insert might be present with use of such an outer cover, as compared with strength required of inside components of a disposable diaper. Therefore, an article such as described herein may include a disposable absorbent insert manufactured from materials that are different from those ordinarily used in the manufacture of disposable diapers, such as petroleum-derived materials, e.g., polyethylene and polypropylene. For example, a disposable absorbent insert having one or more of a topsheet, backsheet, standing cuffs and/or other components formed of products of wood, cotton, flax (linen), hemp, bamboo, or other cellulose fibers (e.g., paper), in addition to the materials identified above, is contemplated. If resistance to aqueous liquid penetration or substantial liquid impermeability is desired, e.g., for a backsheet and/or standing cuffs, a material formed of ordinarily hydrophilic fibers such as paper may be coated or impregnated with a hydrophobic material, such as a skin-compatible oil or wax, to impart the desired resistance to aqueous liquid penetration. Each of the materials forming the insert may be selected so as to be dispersible in water or an aqueous solution, flushable, biodegradable and/or compostable (preferably to an agriculturally usable humus or soil amendment).

Absorbent Core

Turning to FIG. 6, the insert 30 may have an absorbent core 44 disposed within the envelope-like structure formed by the topsheet 32 and backsheet 34. The absorbent core 44 may comprise a wide variety of liquid-absorbent materials 45 commonly used in disposable diapers and other absorbent articles. Examples of suitable absorbent materials include comminuted wood pulp, which is generally referred to as air felt creped cellulose wadding; melt blown polymers, including co-form; chemically stiffened, modified or cross-linked cellulosic fibers; tissue, including tissue wraps and tissue laminates; absorbent foams; absorbent sponges; superabsorbent polymers; absorbent gelling materials (AGM); or any other known absorbent material or combinations of materials. The absorbent core may have an absorbent capacity of at least about 120 g, or at least about 150 g, or at least about 200 g, or from about 120 to about 300 g, reciting for said range every 10 increment therein, as determined by the Core Capacity Test Method herein. In certain embodiments, at least a portion of the absorbent core is substantially cellulose free and contains less than 10% by weight cellulosic fibers, less than 5% cellulosic fibers, less than 1% cellulosic fibers, no more than an immaterial amount of cellulosic fibers or no cellulosic fibers. It should be understood that an immaterial amount of cellulosic material does not materially affect at least one of the thinness, flexibility, and absorbency of the portion of the absorbent core that is substantially cellulose free. Among other benefits, it is believed that when at least a portion of the absorbent core is substantially cellulose free, this portion of the absorbent core is significantly thinner and more flexible than a similar absorbent core that includes more than 10% by weight of cellulosic fibers. The amount of absorbent material, such as absorbent particulate polymer material 45a present in the absorbent core may vary, but in certain embodiments, is present in the absorbent core in an amount greater than about 80%, or greater than about 85%, or greater than about 90%, or greater than about 95% by weight of the core. In nonlimiting examples, a thermoplastic material, such as a thermoplastic adhesive composition 46, may be used to immobilize superabsorbent particles on a substrate (e.g., the topsheet, backsheet or core wrap).

In some embodiments, as shown in FIG. 7A, the absorbent core may comprise one or more channels 47, wherein said channels are substantially free of absorbent particulate polymer material. The channels 47 may extend longitudinally or laterally. The absorbent core may further comprise two or more channels. The channels may be straight, curvilinear, angled or any workable combination thereof. In nonlimiting examples, two channels are symmetrically disposed about the longitudinal axis.

The absorbent core 44 may include a core wrap 48, comprising one or more substrates 49 to encloses the absorbent material 45. Where channels are present, the core wrap may be bonded within one or more channels, thereby providing permanent channels which maintain their channel structure in the wet state.

The absorbent core 44 may be manufactured in a wide variety of sizes and shapes (e.g., rectangular, hourglass, “T”-shaped, etc.). The configuration and construction of absorbent core 44 may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). Exemplary absorbent structures for use as the absorbent core 44 are described in U.S. Pat. Nos. 4,610,678; 4,673,402; 4,834,735; 4,888,231; 5,137,537; 5,147,345; 5,342,338; 5,260,345; 5,387,207; 5,397,316, and U.S. patent application Ser. Nos. 13/491,642 and 15/232,901.

Acquisition-Distribution System

Referring to FIGS. 7A-7B, in some embodiments, an acquisition-distribution system (ADS) 60 is disposed between the topsheet 32 and the absorbent core 44. One function of the ADS is to quickly acquire one or more of the fluids and distribute them to the absorbent core in an efficient manner. The ADS may comprise one, two or more layers, which may form a unitary layer or may remain as discrete layers which may be attached to each other. The ADS 60 may include hydrophilic materials that provide significant wicking of bodily exudates. These materials may dewater the topsheet 32 and quickly move bodily exudates into the absorbent core 44. The ADS 60 may comprise one or more nonwoven materials, foams, cellulosic materials, cross-linked cellulosic materials, air laid cellulosic nonwoven materials, spunlace materials, or combinations thereof, for example. In some instances, portions of the ADS 60 may extend through portions of the topsheet 32, portions of the topsheet 32 may extend through portions of the ADS 60, and/or the topsheet 32 may be nested with the ADS 60. Typically, an ADS 60 may have a width and length that are smaller than the width and length of the topsheet 32. The ADS may have one or more channels 61 as described above with reference to the absorbent core 44. The channels 61 in the ADS may align or not align with channels 47 in the absorbent core 44. In an example, a first acquisition material 62 may comprise a nonwoven material and as second acquisition material 63 may comprise a cross-linked cellulosic material. Suitable ADS are described in WO 2000/59430, WO 95/10996, U.S. Pat. No. 5,700,254, and WO 02/067809, for example.

Cuff Structures

As noted above, the article 10 may comprise a leg gasketing system 50, portions of which may be formed by the insert 30. Returning to FIGS. 4-5, the insert may comprise a pair of longitudinal standing cuffs 54, also referred to as barrier leg cuffs. Each standing leg cuff may be formed by a piece of material which is bonded to the absorbent insert so it may extend upwards from a wearer-facing surface and provide improved containment of fluids and other body exudates approximately at the junction of the torso and legs of the wearer. The standing leg cuffs are delimited by a proximal edge 55 joined directly or indirectly to the topsheet 32 and/or the backsheet 34 and a free terminal edge 56, which is intended to contact and form a seal with the wearer's skin. In some embodiments, the free terminal edge 56 comprises a folded edge. The standing leg cuffs 54 extend at least partially between the front edge 31 and the rear edge 33 on opposite sides of the longitudinal centerline 40 and are at least present in the crotch region.

The standing leg cuffs may be integral with the topsheet 32 or the backsheet 34 or may be a separate material joined to the topsheet and/or backsheet. Each standing leg cuff 54 may comprise one, two or more elastic elements 28 close to the free terminal edge 56 to provide a better seal. The standing cuff may be formed of any of a variety of substrates such as plastic films and woven or nonwoven webs of natural fibers (e.g., wood or cotton fibers), synthetic fibers (e.g., polyester or polypropylene fibers), or a combination of natural and synthetic fibers. In certain embodiments, the standing cuffs may be formed of a substantially liquid impermeable web to contain and isolate liquid exudates from the outer cover, outer clothing and environment of the wearer and/or may be formed of a vapor permeable web for breathability of the insert and article.

Referring to FIGS. 7A-7B, the article 10 may comprise a dual gasketing system, which includes the standing cuffs and gasketing cuffs 57. The gasketing cuffs 57 may be joined to the insert 30, more particularly to the topsheet and/or backsheet. The gasketing cuffs are disposed outboard of the standing cuffs and may provide a better seal around the thighs of the wearer. A gasketing cuff 57 may comprises a proximal edge 58 and a free terminal edge 59. The free terminal edge 59 may comprise a folded edge. Each gasketing cuff may comprise one or more elastic elements 28, which may be sandwiched between other layers of material, such as the portions of material forming the attached proximal portions of the standing cuffs, topsheet, backsheet, separate gasketing cuff material, or combinations thereof. The gasketing cuff may be formed of any of a variety of substrates such as plastic films and woven or nonwoven webs of natural fibers (e.g., wood or cotton fibers), synthetic fibers (e.g., polyester or polypropylene fibers), or a combination of natural and synthetic fibers. As noted with respect to standing cuffs 54, gasketing cuffs may likewise be formed of any suitable web materials but preferably are formed of web materials that are effectively liquid impermeable while being vapor permeable, so as to contain the wearer's liquid exudates within the insert while permitting the insert to “breathe” to avoid excess humidity within the insert (which may overhydrate the wearer's skin and promote conditions such as diaper rash). Suitable materials include nonwoven, films, elastic strands and combinations thereof.

In further embodiments, the leg gasketing system comprises standing leg cuffs that are integral with gasketing cuffs. Suitable leg gasketing systems which may be part of the absorbent article are disclosed in U.S. Pat. App. No. 62/134,622, 14/077,708; U.S. Pat. Nos. 8,939,957; 3, 860,003; 7,435,243; 8,062,279.

Attachment of the Absorbent Insert to the Outer Cover

As noted, the outer cover may also comprise one or more insert fastening components 110I capable of operatively engaging with an insert receiving component 1121 disposed on the absorbent insert, as shown in FIGS. 2-3.

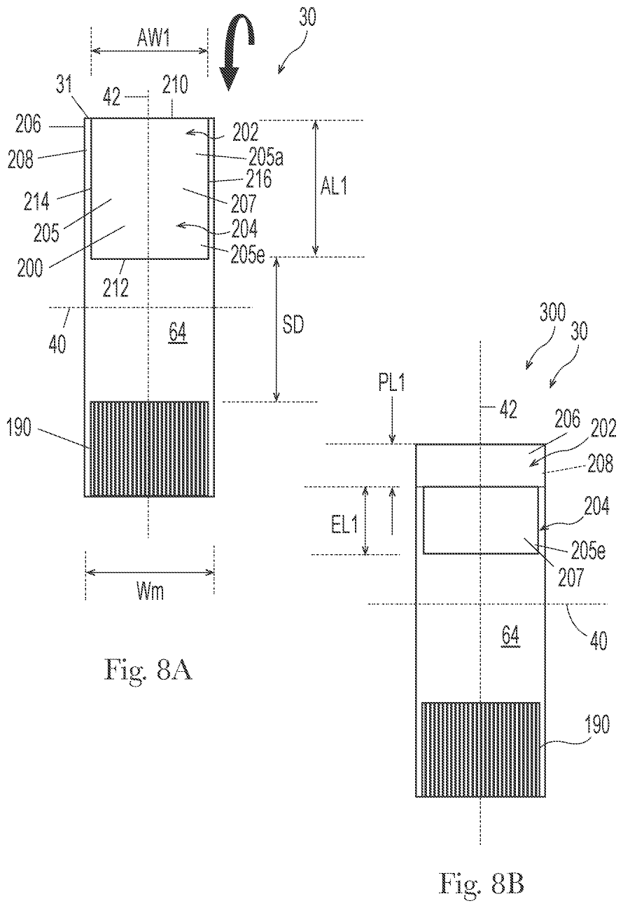

Additionally, or alternatively, the absorbent insert comprises one or more adjustable attachment areas 200 as shown in FIGS. 8A-10B for example. The adjustable attachment area comprises an adjustment portion 202 and an engageable portion 204. The adjustment portion is capable of attaching a first segment 206 of the absorbent insert to a second segment 208 of the absorbent insert. Although the first segment and second segment are shown as longitudinally adjacent, it is also contemplated that that the first segment 206 may be laterally outboard of the second segment 208, such that the attachment results in the insert having a smaller width rather than a shorter length as shown in the figures. The attachment may be formed by any suitable means 205a, including but not limited to adhesive, mechanical fasteners, heat bonding, pressure bonding and combinations thereof. Nonlimiting examples of attachment means include adhesive patch, adhesive strips or tabs, hook and loop fastening components, interlocking fasteners such as tabs & slots, buckles, buttons, snaps, and/or hermaphroditic fastening components. In various embodiments, the attachment means comprises an adhesive 207, more particularly a pressure sensitive adhesive. The adhesive may remain tacky well below its application temperature. For example, a suitable adhesive can be a pressure sensitive hot melt adhesive. Suitable adhesives are disclosed in commonly assigned U.S. Patent App. No. 63/013,573 under attorney docket 15780PQ.

The engageable portion 204 is capable of attaching the absorbent insert to the outer cover. The engageable portion may comprise the same attachment means as the adjustment portion or a different attachment means. Suitable attachment means 205e for the engageable portion include adhesive, mechanical fasteners, heat bonding, pressure bonding and combinations thereof. Nonlimiting examples of attachment means include adhesive patches, adhesive strips or tabs, hook and loop fastening components, interlocking fasteners such as tabs & slots, buckles, buttons, snaps, and/or hermaphroditic fastening components. In various embodiments, the attachment means comprises an adhesive 207, more particularly a pressure sensitive adhesive, even more particularly a pressure sensitive hot melt adhesive.

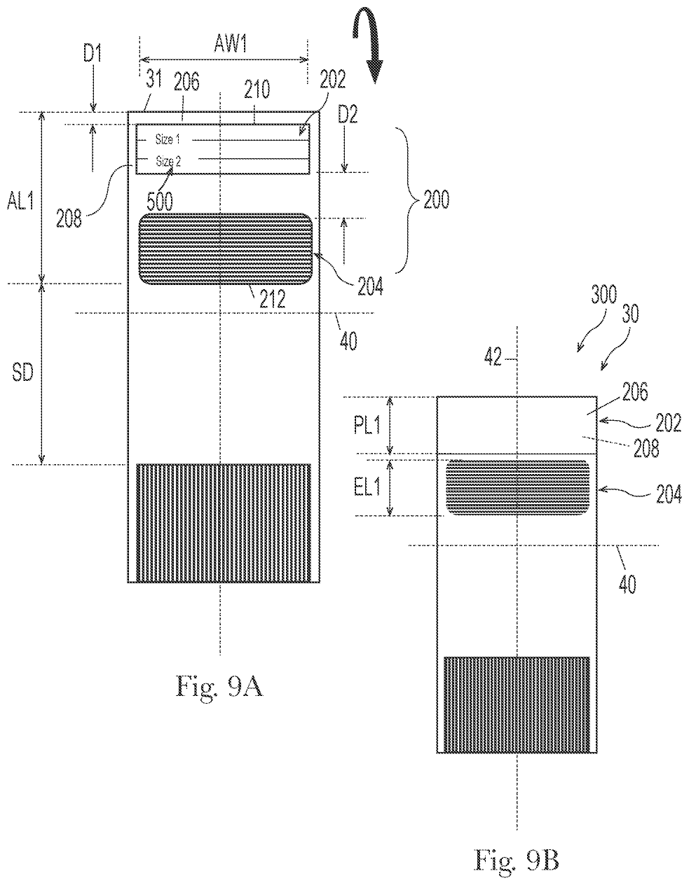

The adjustable attachment area may be disposed on the garment-facing surface 64 of the backsheet. The adjustable attachment area comprises an outboard lateral edge 210 defined by the outboard-most edges of the attachment means, inboard lateral edge 212 defined by the laterally inboard-most edges of the attachment means, and opposing longitudinal edges 214, 216 each defined by longitudinally outboard edges of the attachment means. The outboard lateral edge may be at least partially coterminous with the first lateral end as shown in FIG. 8A. As shown in FIG. 9A, the outboard lateral edge 210 may be separated from the first lateral end 31 by a maximum distance, D1, of about 10 mm or less, or about 5 mm or less, or from about 1 mm to about 10 mm, reciting for said range every 1 mm increment therein. One or both of the longitudinal edges may be at least partially coterminous with the longitudinal edges of the insert 36, 37 as shown in FIG. 3. Alternatively, one or both of the longitudinal edges 214, 216 may be disposed inboard of the respective longitudinal edge(s) of the insert as shown in FIGS. 8A-8B for example.

The adjustable attachment area comprise a maximum length, AL1, extending between the outboard lateral edge and the inboard lateral edge. The maximum length, AL1, may be at least about 40 mm, or at least about 50 mm, or at least about 80 mm, or from about 40 mm to about 150 mm, or from about 60 mm to about 100 mm, reciting for each range every 5 mm increment therein. The maximum length, AL1, may be at least about 20%, or at least about 35%, or at least about 40%, or from about 20% to about 70%, or from about 35% to about 65%, of the length of the absorbent insert, L, reciting for each range every 5% increment therein. In various embodiments, the adjustable attachment portion does not overlap the lateral axis. In this way, the attachment of the outer cover and absorbent insert may be singularized and localized, permitting independent movement of portions of the outer cover and/or absorbent insert. Indeed, by leaving the central portion of the outer cover and absorbent insert unattached, an end region 14, 18 may laterally stretch and contract substantially independently and unimpeded by the structure of the insert and portions thereof. This may avoid lateral bunching of the ends of insert 30 with lateral contraction of the outer cover 20, and/or, conversely, may avoid having the structure of insert 30 restrict outer cover 20 from stretching or contracting laterally, as a result of a more force-coupled arrangement therebetween. It also helps to limiting stress on the absorbent insert in the crotch region, which reduces the risk of lateral bunching between the legs of the wearer.

The adjustable attachment area comprises a maximum width, AW1, extending between the longitudinal edges. The maximum width, AW1, may be at least 50%, or at least 60%, or from about 50% to about 100%, or from about 60% to about 90% of the maximum width of the absorbent insert, Wm, in the area where the adjustable attachment region is present.

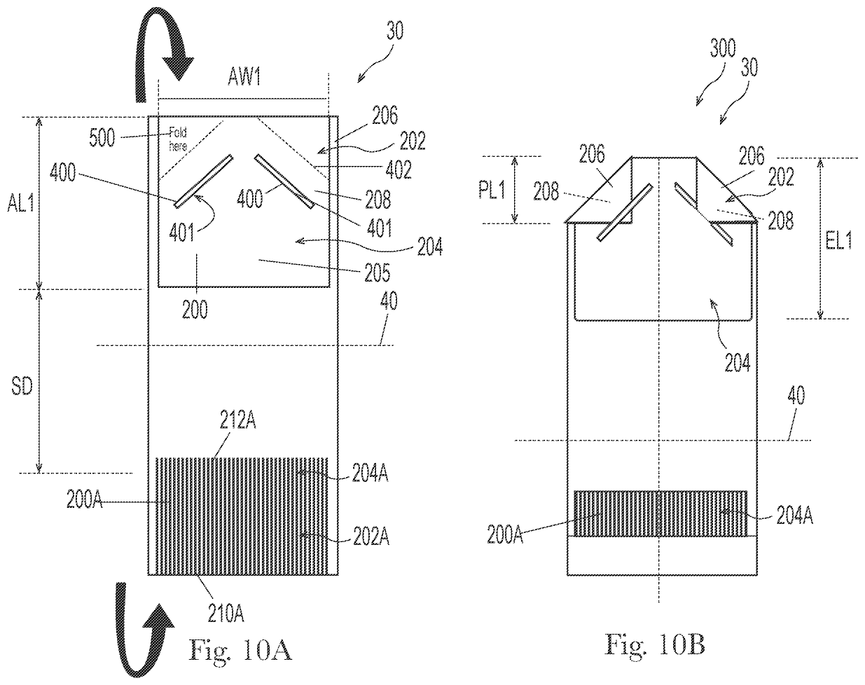

The adjustable attachment portion may be continuous or discontinuous. Where continuous, the attachment means of the engageable portion and the adjustment portion are contiguous as shown in FIG. 8A. Where discontinuous, the attachment means 205 within may be disposed in any suitable noncontiguous shapes, including but not limited to stripes, dots, squares, ellipses and combinations thereof. In nonlimiting examples, the attachment means is disposed in longitudinal stripes, lateral stripes, diagonal stripes or combinations thereof. As shown in the second adjustable attachment area 200A in FIG. 10B, the adjustment portion 202A may be adjacent to the engageable portion 204A and have attachment means contiguous with those of the adjustment portion 202A while the attachment means in the area 200A are discontinuous. Alternatively, the adjustment portion may be separated from the engageable portion when the adjustable attachment portion is discontinuous as shown in FIG. 9A. The adjustment portion may be separated from the engageable portion by a maximum distance, D2, of 3 mm or less, or 1 mm or less, or from about 0.5 mm to about 3 mm, reciting for said range every 0.1 mm increment therein. The adjustment portion and/or the engagement portion may independently be continuous or discontinuous.

The adjustable attachment area may comprise a bending stiffness of about 30 mN or less, or about 25 mN or less, or about 23 mN or less, or from about 10 mN to about 30 mN, or from about 15 mN to about 25 mN as measured by ISO 2493-1-2010, using a 15 degree angle and bending in the longitudinal direction. More particularly, the adjustment portion may comprise bending stiffness of about 30 mN or less, or about 25 mN or less, or about 23 mN or less, or from about 10 mN to about 30 mN, or from about 15 mN to about 25 mN as measured by ISO 2493-1 2010, using a 15 degree angle and bending in the longitudinal direction.

The engageable portion may comprise a maximum length, EL1, of at least about 5 mm, or at least about 10 mm, or at least about 15 mm, or at least about 20 mm, or from about 5 mm to about 100 mm, or from about 10 mm to about 80 mm, or from about 15 mm to about 60 mm, reciting for each range every 5 mm increment therein. Where the engageable portion is contiguous with the adjustment portion, the length of the engagement portion may be measured when the adjustment portion is the attached configuration 300 (i.e., when the first segment is attached to the second segment). The engageable portion may comprise the maximum width, AW1, of the adjustable attachment region. The width of the engageable portion may be less than the maximum width of the adjustable attachment region throughout a portion of its length, EL1, or throughout its entire length, ELL In nonlimiting examples, the engageable portion may be laterally adjacent to the adjustment portion as shown for example in FIG. 10B.

The adjustment portion may comprise a maximum length, PL1, in the attached configuration of at least about 15 mm, or least about 17 mm, or at least about 25 mm, or from about 15 mm to about 80 mm, or from about 20 mm to about 75 mm reciting for each range every 5 mm increment therein. The adjustment portion may comprise the maximum width, AW1, of the adjustable attachment region. The adjustment portion may be less than the maximum width of the adjustable attachment region throughout a portion of its length, PL1, or throughout its entire length, PL1. The adjustment portion may be foldable and may attach the first segment of the insert to the second segment of the insert while in a folded configuration. The adjustment portion may be separable, by for example perforation, and the first segment may be detached from the second segment and then attached thereto. Any suitable means of attaching the first segment to the second segment may be employed. It is to be understood that the first segment need not be continuously attached to the second segment in the attached configuration 300. Further, it is also contemplated that the first segment may be at least partially secured to the second segment using one or more pocket structures 400, wherein the first segment is for example inserted into or through an opening 401 disposed on or near the second segment. In nonlimiting examples, the first segment may be foldable and may have one or more fold lines 402 to facilitate such insertion as shown in FIGS. 10A and 10B.

In embodiments where the adjustable attachment area comprises adhesive, the adhesive coated area may be provided with a protective cover, which is removed prior to use. The protective cover may be a silicone coated release paper, a plastic film or any other easily removable cover. The protective cover may be in a single piece or in a multitude of pieces, e.g. to cover the individual adhesive areas. It also can perform other functions such as providing individualized packaging for the article or provide a disposal function. Any commercially available release paper or film may be used. Suitable examples include BL 30 MG-A SILOX EI/O, BL 30 MG-A SILOX 4 P/O available from Akrosil Corporation, and M&W films available from Gronau in Germany, under the code X-5432.

The absorbent insert 30 may comprise a second attachment area 190 as shown for example in FIGS. 8A-8B. The second attachment area may be capable of attaching the absorbent insert to the outer cover and/or attach the absorbent insert to itself. Any suitable attachment means described above may be utilized. The attachment means may be disposed continuously or discontinuously. In various embodiments, the second attachment area comprises a second adjustable attachment area 200A as shown in FIG. 10A. The second adjustable area comprises an adjustment portion 202A and an engageable portion 204A and may have any of the features described above with respect to the adjustable attachment area, including the dimensions. The second attachment area is separated from the adjustable attachment region 200 by a minimum separation distance, SD, of at least about 25 mm, or at least about 50 mm, or at least about 150 mm, or about 200 mm, or from about 10 mm to about 300 mm, or from about 100 mm to about 250 mm, or from about 150 mm to about 200 mm, reciting for each range every 5 mm increment therein. An outboard lateral edge 210A may be at least partially coterminous with the second lateral end 33 of the absorbent insert. The outboard lateral edge 210A of the second attachment area may be disposed within 10 mm or less, or about 5 mm or less, or from about 1 mm to about 10 mm, of the second lateral end of the insert 33, reciting for said range every 1 mm increment therein.

The adjustable attachment area 200 and the second attachment area 190 may differ by one of the group consisting of: peel strength, type(s) of attachment means, peripheral shape of the attachment areas, surface area, length, width, whether continuous and discontinuous, spacing between attachment means in discontinuous dispositions, disposition patterns of attachment means, the dimensions of the engageable portions (as applicable), the dimensions of adjustable portions (as applicable) and combinations thereof.

The article may comprise instructional indicia 500 providing guidance or instruction to the caregiver relative to use of the adjustable attachment area (more particularly where to fold or otherwise shorten the absorbent insert), placement of the insert into the outer cover, and/or placement and/or fit of the article about the wearer. Instructional indicia may be in the form of words, letters, pictures, lines (e.g., demarcating proper alignment), notches (i.e., cuts or indentations), creases, matching patterns/colors/indicia between an absorbent insert and an outer cover and combinations thereof. The instructional indicia may be disposed on the outer cover as shown in FIG. 2 and/or on the absorbent insert as shown in FIGS. 9A and 10A. The instructional indicia may be disposed in or proximate to an adjustable attachment area 200.

### Bio-Sourced Materials

Components of the disposable absorbent article can at least partially be comprised of bio-sourced content as described in U.S. Pat. Pub. Nos. 2007/0219521A1, 2011/0139658A1, 2011/0139657A1, 2011/0152812A1, and 2011/0139659A1. These components include, but are not limited to, topsheets, backsheet films, backsheet nonwovens, side panels, leg gasketing systems, superabsorbent, acquisition layers, core wrap materials, adhesives, outer covers, fastener systems, and landing zones. In at least one embodiment, a disposable absorbent article component comprises a bio-based content value from about 10% to about 100%, or from about 25% to about 75%, or from about 50% to about 60% using ASTM D6866-10, method B. In order to apply the methodology of ASTM D6866-10 to determine the bio-based content of any component, a representative sample of the component must be obtained for testing. In at least one embodiment, the disposable absorbent article component can be ground into particulates less than about 20 mesh using known grinding methods (e.g., WILEY® mill), and a representative sample of suitable mass taken from the randomly mixed particles.

Core Capacity Test Method

The Centrifuge Retention Capacity (CRC) is a measure of the fluid retention capacity of a specimen submerged in 0.9% NaCl saline solution for 30 minutes and then subjected to centrifugation. The test is a based on the Worldwide Strategic Partners Standard Test Method WSP 241.2 (09), Gravimetric Determination of Fluid Retention Capacity in Sainte Solution after Centrifugation.

For analysis, specimens are placed into a bag constructed from heat-sealable, water-permeable, non-apertured nonwoven. Specifications for the bag material are given in WSP 241.2 (09). Measure the length (L) and width (W) of the specimen to be tested. Cut a piece of the bag material that is 2×L+50 mm by W+25 mm. Fold the bag material in half across its width and heat-seal two of the open edges approximately 3 to 5 mm from the edges. The finished bag is L+25 mm by W+25 mm. In addition to the bags for samples, prepare three bags of the same dimensions to be used as blanks.

Absorbent Insert Sample Preparation: Precondition samples at about 23° C.±2° C. and about 50%±2% relative humidity for 2 hours prior to testing. The absorbent insert is unfolded and placed with the wearer-facing surface facing upward, Using scissors cut any elastic along the longitudinal edges of the article at an interval of approximately 2 cm, such that the article can be laid flat. Lay the absorbent insert on a piece of aluminum foil and cut the absorbent insert along the longitudinal and lateral axis resulting in four individual specimens. On a calibrated balance, tare the weight of an appropriately sized bag. Place the specimen, and any material that fell onto the foil during cutting, into the tarred bag and heat seal the remaining open edge. Obtain and record the dry mass of the specimen to the nearest 0.001 grams. Repeat this procedure to obtain the dry mass of the remaining specimens. Three replicate absorbent inserts are prepared for testing.

### Test Procedure

Obtain a pan large enough to hold several bags or specimens, and fill it with 0.9% saline solution to a level such that the bag or specimen may be completely submerged. Lay the bags or specimens to be tested onto the surface of the saline, and allow them to become wet for 1 minute before submerging. A weight placed onto the edge of the bag may be used to ensure that the sample remains submerged while allowing the solution to be freely absorbed by the specimen. After 30 minutes remove the bag or specimen from the saline solution, and immediately transfer it into the basket of a suitable centrifuge capable of subjecting the specimens to a 250 G centrifugal acceleration (such as a Clay Adams Dynac Centrifuge available from Block Scientific, Bohemia, N.Y.). Orient the specimens in the centrifuge such that the wearer-facing surface is facing outward, unless the wearer-facing surface is hydrophobic, in which case it should be oriented inward. Position the bags or specimens so that similarly weighted samples are opposite each other for proper balancing. Centrifuge the bags or specimens for 3 minutes±10 seconds at 250 G. The bags or specimens are then removed and immediately weighed. Record the wet mass of the specimen and bag, blank bag, or specimen without a bag to the nearest 0.001 grams.

Calculate the average of the three wet blank bag masses after centrifugation (m_{b}); this value is disregarded if the specimen was tested without a bag. For each specimen (i=1, 2, and 3), calculate the centrifuge retention capacity (wᵢ) expressed as a mass fraction (g/g) using the following equation:

wᵢ=((m_{wi}−m_{b})−mₛᵢ)/mₛᵢ

Where me is the mass, expressed in grams, of the dry specimen and m_{wi} is the mass, expressed in grams, of the wet specimen and bag or specimen without a bag. For the absorbent inserts cut and tested as four separate specimens. Sum up the four individual masses of the dry specimens to get mₛᵢ, sum up the four individual masses of the wet specimens and bags to get m_{wi}, and sum up four of the average wet blank bag masses after centrifugation to get m_{b}. Use these values to calculate a total centrifuge retention capacity (wᵢ) value. Calculate and report the average centrifuge retention capacity for the three replicates to the nearest 0.001

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
an absorbent insert and an outer cover, wherein the absorbent insert comprises:
a topsheet, a backsheet, and an absorbent core disposed between the topsheet and backsheet;
a longitudinal axis and a lateral axis;
a first lateral end and a second lateral end; and
an adjustable attachment area having an adjustment portion and an engageable portion,
wherein the adjustment portion is capable of attaching a first segment of the absorbent insert to a second segment of the absorbent insert and wherein the engageable portion is capable of attaching the absorbent insert to the outer cover.

2. The absorbent article of claim 1 wherein the adjustable portion comprises an bending stiffness of 30 mN or less, according to ISO 2493-1-2010.

3. The absorbent article of claim 1 wherein the adjustable attachment area has outboard lateral edge that is within 10 mm of the first lateral end and wherein the adjustable attachment area does not overlap the lateral axis.

4. The absorbent article of claim 3 wherein the outboard lateral edge is at least partially coterminous with the first lateral end.

5. The absorbent article of claim 1 wherein the adjustable attachment area comprises a length, AL1, of 40 mm to 150 mm.

6. The absorbent article of claim 1 wherein the adjustable attachment area comprises a maximum width, AW1, that is 50-100% of a maximum width of the absorbent area where the adjustable attachment area is present, Wm.

7. The absorbent article of claim 1 wherein the absorbent insert comprises a length L, and the adjustable attachment area has a length, AL1, that is between 20% and 70% of L.

8. The absorbent article of claim 1 wherein the absorbent insert is disposable and the outer cover is reusable.

9. The absorbent article of claim 1 wherein the adjustable attachment area comprises adhesive.

10. The absorbent article of claim 1 wherein the adjustable attachment area is discontinuous.

11. The absorbent article of claim 10 wherein the adjustable attachment area is disposed in longitudinal stripes, lateral stripes, or combinations thereof.

12. The absorbent article of claim 1 wherein the engageable portion has a longitudinal length, EL1, of at least 10 mm.

13. The absorbent article of claim 1 wherein the absorbent insert and/or the outer cover further comprise instructional indicia.

14. An absorbent article comprising:
an absorbent insert and an outer cover, wherein the absorbent insert comprises:
a topsheet, a backsheet, and an absorbent core disposed between the topsheet and backsheet;
a longitudinal axis and a lateral axis;
a first lateral end and a second lateral end, and a length, L extending between the first and second lateral ends;
a first adjustable attachment area having a length AL1, that is between 20% and 70% of L, wherein the first adjustable attachment area comprises an adjustment portion and an engageable portion, wherein the adjustment portion is capable of attaching a first segment of the absorbent insert to a second segment of the absorbent insert and wherein the engageable portion is capable of attaching the absorbent insert to the outer cover; and
a second attachment area, wherein the first adjustable attachment area and the second attachment area are separated by a distance of at least 50 mm.

15. The absorbent article of claim 14 wherein the first adjustable attachment area and the second attachment area differ.

16. The absorbent article of claim 14 wherein the second attachment area is adjustable.

17. An adjustable absorbent insert comprising:
a first lateral end and a second lateral end, and a length, L extending between the first and second lateral ends; and
an adjustable attachment area comprising:
an outboard lateral edge disposed within 10 mm of the first lateral end;
an adjustment portion capable of attaching a first segment of the absorbent insert to a second segment of absorbent insert and comprising a length, AL1, that is between 20% and 70% of the length, L; and
an engageable portion;
wherein in an attached configuration, the engageable portion comprises a length, EL1, of at least 10 mm.

18. The absorbent article of claim 17 wherein the adjustment portion comprises an bending stiffness of 30 mN or less, according to ISO 2493-1-2010.

19. The absorbent insert of claim 17 wherein the adjustment portion at least partially comprises adhesive.

20. The absorbent insert of claim 17 wherein the engageable region does not overlap a lateral axis of the absorbent insert.
